(19) United States
(12) Patent Application Publication
(10) Pub. No.: US 2006/0149342 A1
(43) Pub. Date: Jul. 6, 2006

(54) Light therapeutic device

(75) Inventors: Sheng Bang Huang, Changhua (TW); Ming I Lee, Taipei (TW); Sun Bin Yin, Hsinchu (TW); Chun Wei Wang, Taipei (TW); Yih-Chih Hsu, Taipei (TW); Jung-Tsung Hsu, Hsinchu (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/074,724

(22) Filed: Mar. 9, 2005

(30) Foreign Application Priority Data
Dec. 30, 2004 (TW) 93141350

(51) Int. Cl. A61N 5/06

(52) U.S. Cl. 607/88

(57) ABSTRACT

A light therapeutic device. The light therapeutic device comprises a flexible optical element capable of deforming corresponding to an affected part of a patient and providing a light of specific wavelength for treating the affected part. The flexible optical element comprises a flexible optical conductor coated with opaque material, and light is emitted from the flexible optical conductor by selectively removing the opaque material of a part of the optical conductors.

### BACKGROUND

The invention relates to a light therapeutic device, and in particular to a light therapeutic device with a flexible optical element capable of deforming corresponding to an affected part and providing light of specific wavelengths for treatment.

Traditionally, oral tumors have been removed by surgical operation with the aid of radiotherapy and chemical therapy. Such an intrusive treatment often causes serious problems in speaking, chewing, swallowing and even loss of taste. Thus a treatment method without the described problems of the conventional method is desirable. Photodynamic therapy (PDT) employs certain light sensitive material exposed under light of specific wavelength to kill cells. As a tumor cell can absorb more light sensitive material than a normal cell, PDT is more effective in treatment of oral tumors than the conventional method. As the pain of surgery, radiotherapy or chemical therapy can be prevented by use of such treatment, it has been highly valued recently. For the past ten years, as the new light sensitive material and laser apparatus have been developed, and medical professionals have paid great attention to the PDT, and FDA, Europe and Japan have applied the PDT to clinical experiments on tumor treatment.

The light source of the PDT permitted by FDA is a laser source of single wavelength. As the costs of purchase and maintenance of a laser source are, however, quite high, and as such PDT is not popularly used. As light emitting diodes (LEDs) have lower weight, low cost and reduced power consumption, they can replace the laser source to serve as the light source of a PDT.

### SUMMARY

A light therapeutic device according to an embodiment of the invention comprises a flexible optical element deforming corresponding to the shape of an affected part and providing light with specific wavelengths for treatment of the affected part. The therapeutic light can, thereby, reach cancer organisms and uniformly provide therapeutic energy for the affected part.

Preferably, the flexible optical element can be a light such as an organic light emitting diode (OLED).

Preferably, the flexible optical element is sheet-shaped, and a desired light intensity is available by overlapping a plurality of the flexible optical elements.

Preferably, the light therapeutic device further comprises a light source and an optical coupler. The light from the light source is directed into the flexible optical element via the optical coupler. The flexible optical element comprises a flexible optical conductor coated with opaque material, and light is emitted from the flexible optical conductor by selectively removing the opaque material of a part of the flexible optical conductors. The flexible optical conductor can be an optical textile comprising a plurality of optical fibers.

Preferably, light from the light source is directed from single side or multiple sides of the flexible optical conductor via the optical coupler. In the case of single sided incidence, a reflector or a reflective layer is disposed on another side of the optical conductor to reflect light back to the flexible optical conductor.

Preferably, the light source can be an incandescent lamp, a cathode fluorescent lamp or a light emitting diode.

### BRIEF DESCRIPTION OF THE DRAWINGS

The invention can be more fully understood by reading the subsequent detailed description and examples with references made to the accompanying drawings, wherein:

FIG. 1 is a schematic view of an embodiment of the light therapeutic device according to the invention;

FIG. 2 is an enlarged view of an optical fiber textile of FIG. 1;

FIG. 3 is a schematic view of another optical fiber textile of the invention;

FIGS. 4a, 4b are another embodiment of the light therapeutic device according to the invention;

FIG. 5 is a schematic view depicting the optical fiber textile of FIG. 2 for treatment of an oral tumor inside an oral cavity;

FIG. 6 is a schematic view depicting the optical fiber textile of FIG. 2 for treatment of an oral tumor outside an oral cavity;

FIG. 7 is a picture of an optical fiber textile of the invention; and

FIG. 8 is a picture of a luminous optical fiber textile of the invention.

### DETAILED DESCRIPTION

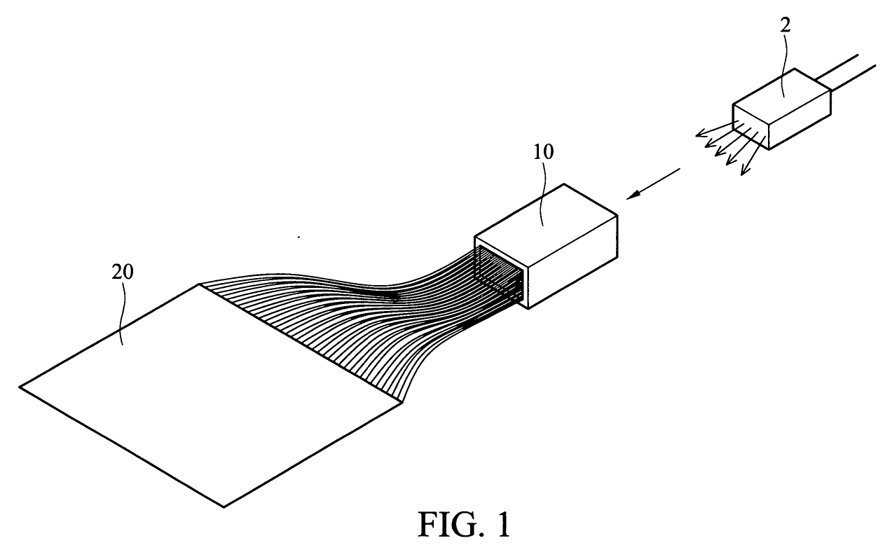

FIG. 1 is a schematic view of an embodiment of the light therapeutic device according to the invention. Light from a light source 2 is directed to a flexible optical fiber textile (flexible optical element) via an optical coupler 10.

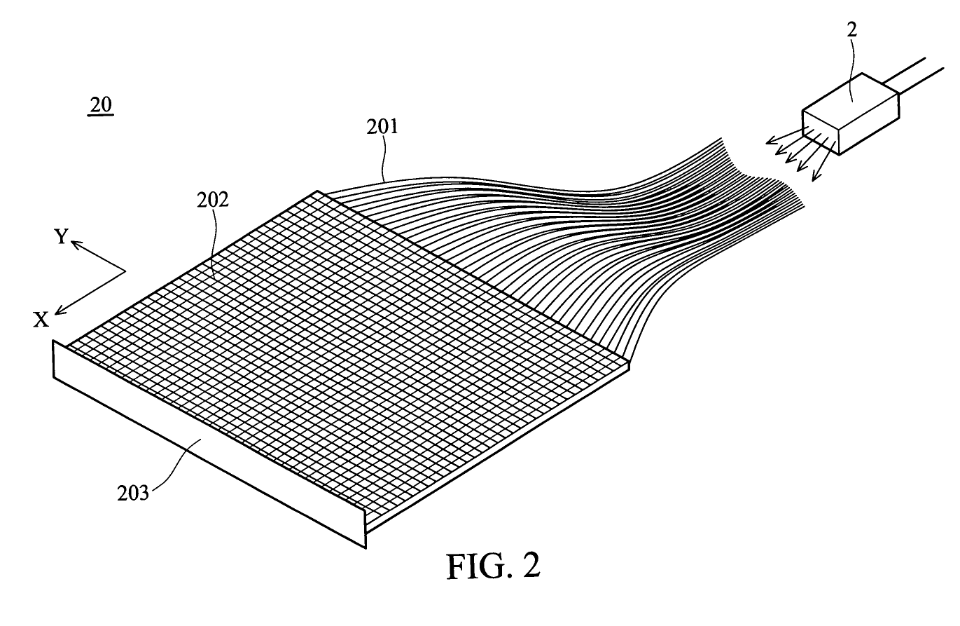

FIG. 2 is an enlarged view of an optical fiber textile of FIG. 1. The optical fiber textile 20 comprises a plurality of optical fibers 201 extending in the x direction and a plurality of weave threads 202 crossing the optical fibers 201 and extending in the y direction.

In general, the optical fibers 201 are coated with opaque material which prevent light from escaping from peripheries of the optical fibers 201. In the invention, however, as the light is desired to emit from the peripheries of the optical fibers 201, the opaque material of a part of the optical fibers 210 is removed. The light entering the optical fibers 201 is emitted through the peripheries of the optical fibers 201 without opaque material to an affected part of a patient. The number of optical fibers without opaque material is adjustable according to treatment requirements.

Referring to FIG. 2, light from the light source 2 is directed into the optical fiber textile 20 from a single side thereof. A part of the incident light is emitted from the peripheries of the optical fibers 201 without opaque material, and the other incident light emits from the end of the optical fiber 201. Therefore, a reflective layer (metal with high reflectivity) or a reflector 203 is required on the end of the optical fiber 201 to reflect light back to the optical fiber 201 The reflector 203 comprises flexible material such as rubber or plastic coated with metal. Thus most of the incident light is emitted from the peripheries of the optical fibers 201 without opaque material.

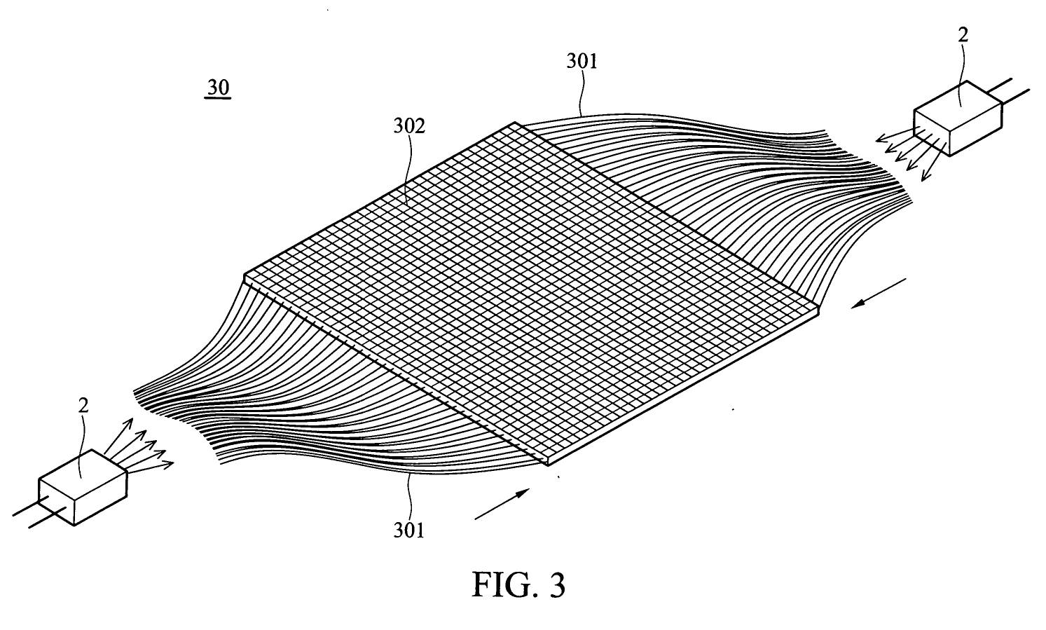

FIG. 3 depicts another optical fiber textile of the invention. Optical fiber textile 30 has a similar structure as the optical fiber textile shown in FIG. 2. Optical fiber textile 30 comprises a plurality of optical fibers 301 and a plurality of weave threads 302 crossing the optical fibers 301. In FIG. 3, light from light source 2 is, however, directed from two sides of the optical fiber textile 30, and emits from the peripheries of the optical fibers 301 without opaque material.

In addition to an external light source, the light source can also be disposed inside the flexible optical element.

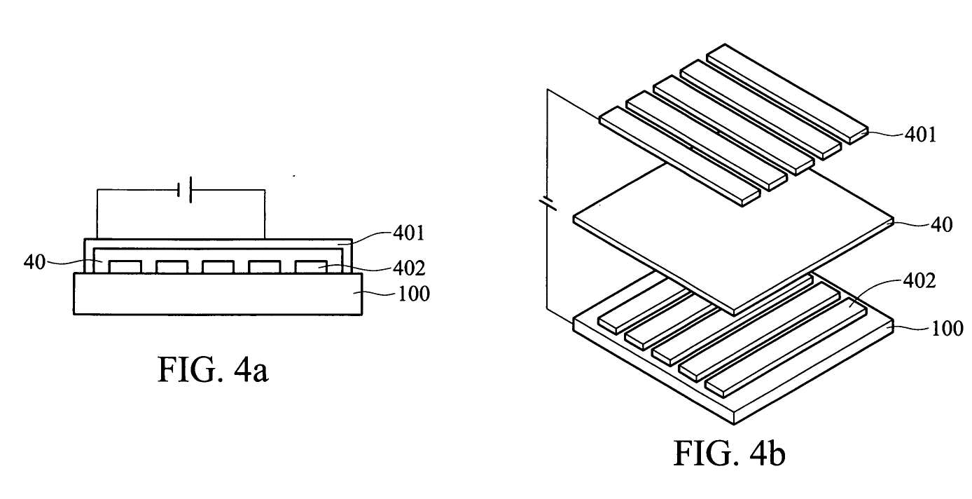

FIGS. 4a and 4b depict another embodiment of the invention. An organic light emitting diode layer (OLED layer) 40 is disposed on a transparent substrate 100 to serve as a light source. A metal electrode 401 is disposed on the OLED layer 40. A transparent electrode 402 is disposed between the OLED layer 40 and the transparent substrate 100 such that the OLED layer 40 can emit light. As the elements described above are flexible, they can deform corresponding to the shape of the affected part.

In this embodiment, the flexible optical element has a built-in light source, and no external light source is needed.

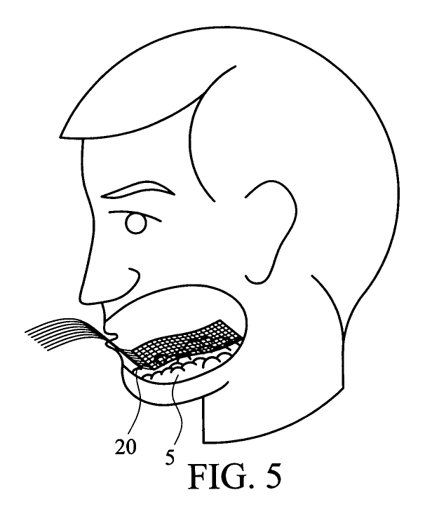

FIG. 5 depicts the optical fiber textile 20 of FIG. 2 used in an oral cavity of a patient. The optical fiber textile 20 is placed in an oral cavity of a patient to cover an affected part 5. Light is directed into the optical fiber textile 20 for treatment of the affected part 5.

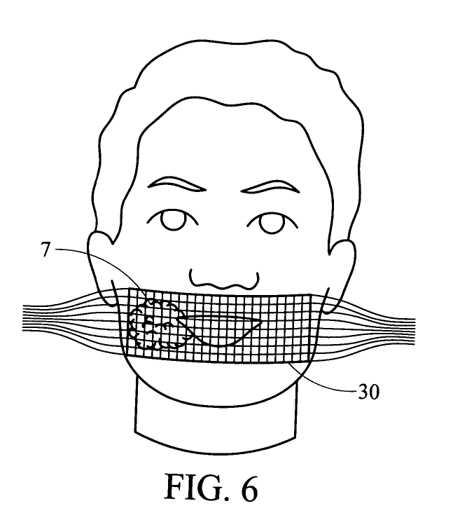

FIG. 6 depicts the optical fiber textile 30 of FIG. 3 used in an external affected part. The optical fiber textile 30 is directly placed on the affected part, and light is directed into the optical fiber textile 30 for treatment.

In addition to the fact that the number of optical fibers without opaque material (light emitting area) can be varied, the light intensity of the light source can also be adjusted, or several optical fiber textiles 20 can be overlapped to obtain different light intensities for various treatment conditions.

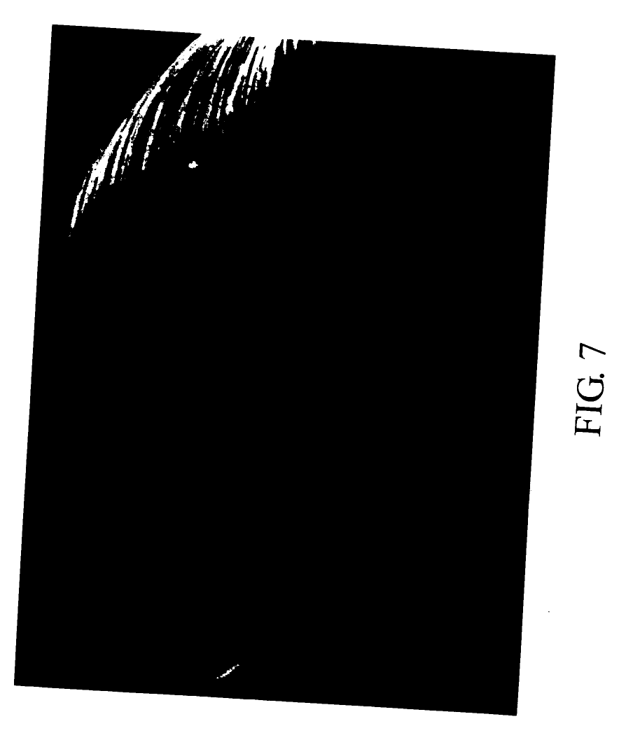

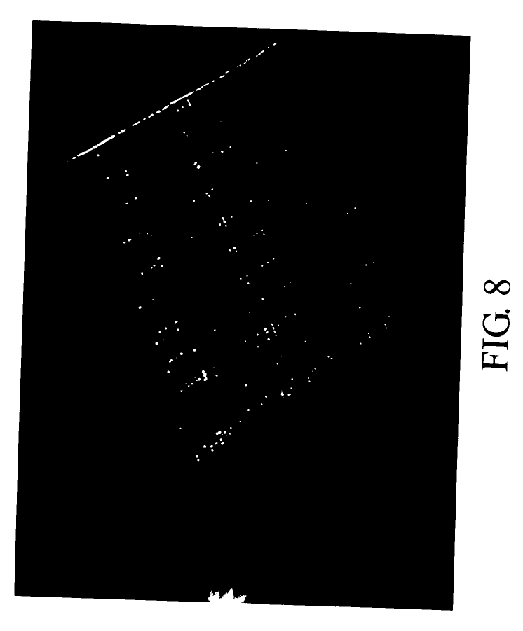

FIG. 7 is a picture of an actual optical fiber textile 20. FIG. 8 is a picture shows the optical fiber textile of FIG. 7 emitting light.

The external light source 2 can be an incandescent lamp, a cathode fluorescent lamp or a light emitting diode. The light coupler 10 can be a lens, a lens duct or similar.

The flexible optical fiber of the invention can be applied to affected parts of various shapes. It is adaptable to an affected part in the depth of an oral cavity where a conventional light therapeutic device cannot reach. The light from the optical fiber textile has a planar distribution which is adapted for the affected part of a large area.

The optical fiber textile can be held in a mouth of a patient so that the patient does not need keep his mouth opened. The optical fiber textile of the invention is held in the mouth to prevent secretion of saliva which dilutes light sensitive material. The light therapeutic device of the invention needs not to be held by a doctor thus one doctor can treat many patients simultaneously.

The light therapeutic device of the invention is not only applicable in the treatment of cancer. A strong LED source can also be helpful for wound recovery and for muscle aches. The light therapeutic device of the invention has various applications for medical treatments.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A light therapeutic device for an affected part, comprising:
a flexible optical element deforming corresponding to the shape of the affected part and providing light with specific wavelengths for treatment of the affected part.

2. The light therapeutic device as claimed in claim 1, wherein the flexible optical element comprises a built-in light source.

3. The light therapeutic device as claimed in claim 2, wherein the built-in light source is an organic light emitting diode.

4. The light therapeutic device as claimed in claim 1, wherein the flexible optical element is sheet-shaped, and a desired light intensity is available by overlapping a plurality of the flexible optical elements.

5. The light therapeutic device as claimed in claim 1 further comprising a light source and an optical coupler, wherein light from the light source is directed into the flexible optical element via the optical coupler.

6. The light therapeutic device as claimed in claim 5, wherein the flexible optical element comprises a flexible optical conductor coated with opaque material, and light is emitted from the flexible optical conductor by selectively removing the opaque material of a part of the optical conductors.

7. The light therapeutic device as claimed in claim 6, wherein the flexible optical conductor is an optical textile comprising a plurality of optical fibers.

8. The light therapeutic device as claimed in claim 6, wherein light from the light source is directed into the flexible optical conductor from single side thereof via the optical coupler.

9. The light therapeutic device as claimed in claim 8 further comprising a reflector disposed on another side to reflect light back to the flexible optical conductor.

10. The light therapeutic device as claimed in claim 8 further comprising a reflective layer formed on another side of the flexible optical conductor.

11. The light therapeutic device as claimed in claim 6, wherein light from the light source is directed into the flexible optical conductor from multiple sides thereof via the optical coupler.

12. The light therapeutic device as claimed in claim 5, wherein the light source is an incandescent lamp.

13. The light therapeutic device as claimed in claim 5, wherein the light source is a cathode fluorescent lamp.

14. The light therapeutic device as claimed in claim 5, wherein the light source is a light emitting diode.
